(19) United States
(12) Patent Application Publication
(10) Pub. No.: US 2015/0209470 A1
(43) Pub. Date: Jul. 30, 2015

(54) Oxygenated Oil Ointment

(71) Applicant: John D. Young, Seminole, FL (US)

(72) Inventor: John D. Young, Seminole, FL (US)

(21) Appl. No.: 14/607,665

(22) Filed: Jan. 28, 2015

Related U.S. Application Data
(60) Provisional application No. 61/932,555, filed on Jan. 28, 2014.

(51) Int. Cl. A61L 26/00

(52) U.S. Cl. CPC A61L 26/0057; A61L 26/0004; A61L 26/0066; A61L 2300/10; A61L 2300/404; A61L 2300/22

(57) ABSTRACT

An oxygenated oil ointment is made, for example, by stirring or agitating plant-based oil while injecting a gas into the plant-based oil, resulting in a coagulated ointment that is beneficial for topical application. In one example, the plant-based oil is olive oil and the gas includes ozone, which is known to kill pathogens. For some plant-based oils, the ozone increases amounts of peroxide in the plant-based oils. Once the olive oil coagulates with the ozone, the resulting ointment retains some of the ozone gas until the ointment is applied on the skin for treatment of, for example, cuts and sores. In some ointments, the pH of the plant-based oil is increased by adding one or more alkaline materials to the plant-based oil before coagulation occurs. This increase in alkalinity improves the ointment's ability to give off ozone and oxygen.

### CROSS-REFERENCE TO RELATED APPLICATION

This application claims the benefit of U.S. provisional application No. 61/932,555 filed on Jan. 28, 2014, the disclosure of which is incorporated by reference.

### FIELD

This invention relates to the field of skin care and more particularly to an ointment comprising oxygenated plant oil.

### BACKGROUND

The application of many plant oils (oils from the nut, seed, or other part of a plant) to skin is known. Many lotions contain oils from plants such as olive oil, jojoba oil, almond oil, coconut oil, grape seed oils, etc.

Applying of such oils directly on the skin is often unappealing because of the room temperature (approximately 70 degrees F.) consistency of the oils. Furthermore, although medical benefits are known for some such oils, little has been done to incorporate such plant-based oils into a medication that helps in wound care and healing.

Petroleum jelly has long been used for wound and skin care, providing a convenient method of application and sustaining skin moisture, especially when applied to dry, cracking skin as often occurs in arid climates. Petroleum jelly is a vacuum distillation of hydrocarbons resulting in a gel that has a melting point usually within a few degrees of human body temperature. Although often used for wound care, studies have shown that petroleum jelly has no medicinal effect. Some petroleum jelly includes phenol (carbolated petroleum jelly) for providing antibacterial effect, but such products have been discontinued.

What is needed is an ointment comprising oxygenated plant oil for various uses such as the care and healing of a wound.

### SUMMARY

In one embodiment, an oxygenated oil ointment is made by stirring or agitating plant-based oil (e.g., olive oil, hemp oil, sunflower oil, jojoba oil, or a combination of such) while injecting a gas, resulting in a coagulated ointment that is beneficial for topical application. In one example, the plant-based oil is olive oil and the gas includes ozone (or predominately ozone), the ozone is known to kill pathogens. Once the ozone coagulates with the olive oil, the resulting ointment retains the ozone gas until the ointment is applied on the skin for treatment of, for example, cuts and sores. The pH of the ointment is increased by adding one or more alkaline materials to the plant-based oil before coagulation occurs. This increase in alkalinity improves the ointment's ability to give off ozone and oxygen to assist in wound healing.

In another embodiment, a coagulated composition for topical application is disclosed including, a plant-based oil mixed with ozone gas until coagulation occurs. For example, olive oil mixed with a gas that includes ozone (O₃) until the olive oil coagulates, thereby retaining some of the ozone (O₃) within the coagulated olive oil and generating an amount of peroxides.

In another embodiment, a method of making a coagulated composition for topical application includes mixing a mixture of 750 milliliters ultra-pure water having an electrical resistance of 16-26 megohms-cm with 330 milliliters of calcium chloride, 660 milligrams of magnesium sulfate, 47,500 milligrams of Sodium silicate pentahydrate, and 1000 milligrams sodium benzoate, then heating the mixture to 80 degrees Celsius for ten minutes. The mixture is then cooled for ten minutes at room temperature (approximately 70 degrees F.), then 100 milliliters of sulfated castor oil is added to the mixture and the mixture is heated to 90 degrees Celsius for approximately one hour. Next, the mixture is added to an amount of plant-based oil into a mixing container. The mixture is added to the plant-based oil at a rate of one teaspoon (4.9289 ml) of the mixture per 32 ounces (0.946353) of the plant-based oil. Now, a gas (preferably ozone O₃ or a mixture of gases that are predominately ozone) is continuously injected into the plant-based oil at a location below a surface of the plant-based oil until the plant-based oil coagulates.

### BRIEF DESCRIPTION OF THE DRAWINGS

The invention can be best understood by those having ordinary skill in the art by reference to the following detailed description when considered in conjunction with the accompanying drawings in which:

FIG. 1 illustrates a schematic view of an exemplary system for oxygenating plant oil.

### DETAILED DESCRIPTION

Reference will now be made in detail to the presently preferred embodiments of the invention, examples of which are illustrated in the accompanying drawings. Throughout the following detailed description, the same reference numerals refer to the same elements in all figures.

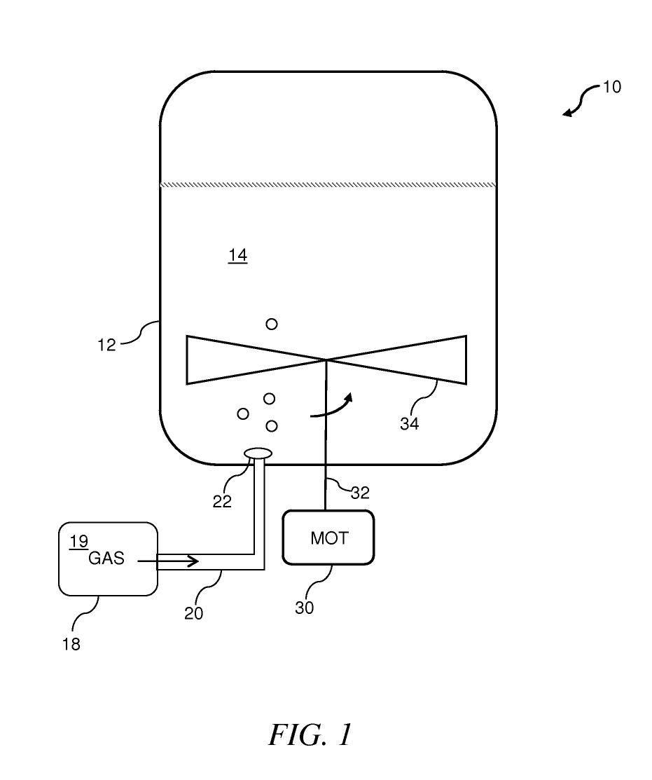

Various plant-based oils 14 (see FIG. 1) are believed to be beneficial when applied to the skin. For example, many apply olive oil directly on their face to reduce effects of aging, etc. Application of such plant-based oils 14 is not easy as these plant-based oils 14 are generally liquid at room temperature (approximately 70 degrees F.). To overcome this problem, the plant-based oils 14 are stirred or agitated while a gas 19 is added, thereby coagulating the plant-based oils 14. The gas 19 is preferably oxygen or ozone (O3), though other gases or combinations of gases are anticipated such as nitrogen, air, etc. Some combinations of gases are also expected for any reasons. It is often difficult to obtain pure oxygen and/or ozone, so it is anticipated that the gas 19, in some embodiments, is primarily oxygen (O₂) and/or primarily ozone (O₃). During the mixing process, the gas molecules (e.g. O₃ molecules) become encapsulated in the plant-based oil 14, preventing escape and/or interaction with other gases such as air.

Referring to FIG. 1, a schematic view of an exemplary system 10 for adding the gas 19 and coagulating the plant-based oils 14 with a gas 19 is shown. The system 10 includes a mixing container 12 in which the plant-based oil 14 is placed and a source (e.g. tank 18) of a gas 19 such as air, oxygen (O₂), ozone (O₃), etc. To mix the gas 19, the gas 19 is routed under pressure from the source of gas 19 into the mixing container 12 through plumbing 20 and, optionally, a one-way check valve 22. As the gas 19 enters the plant-based oil 14, in some embodiments, the plant-based oil 14 is stirred by, for example, a blade 34 mounted to a shaft 32 that is coupled to a drive device such as a motor 30. The process continues until the plant-based oil 14 coagulates into a gelatinous state similar to that of petroleum jelly. Once the plant-based oil 14 coagulates, the coagulated plant-based oil 14 is removed from the mixing container 12 and packaged. In some embodiments, the above process is performed in a chilled environment and/or the resulting coagulated plant-based oil 14 is also refrigerated until used.

Although any plant-based oil 14 is anticipated, oils from olives (olive oil), sunflowers (sunflower oil), and Jojoba (Simmondsia chinensis), known as Jojoba oil are known to work well.

Olive oil is known to improve skin complexion. Jojoba oil is known to be a fungicide.

In a preferred embodiment, the gas 19 is ozone (O₃) or a gas 19 that at a minimum contains some percentage of ozone (O₃). Ozone (O₃) is known to kill pathogens, including pathogens that cause Methicillin-resistant Staphylococcus aureus (MRSA). Ozone (O₃), in its natural state, is very unstable and breaks down into diatomic allotrope, O₂. When ozone (O₃) is suspended in the plant-based oil 14, it becomes more stable and available to help reduce pathogens when applied to skin, particularly in the area of a wound. As the ozone (O₃) breaks down into O₂ and O₁, the O₂ feeds the skin and wounds with O₂, thereby assisting in healing while the O₁ results in lowering the pH of the ointment. To balance the acidity resulting from the O₁, in some embodiments a source of alkalinity is added to the plant-based oil 14 before mixing and coagulation.

In some embodiments, to improve the effectiveness of the Ozone (O₃), the pH of the plant-based oil 14 is adjusted to increase the pH by adding high pH materials to the plant-based oil 14, preferably before coagulation occurs. For example, any combination of one or more of the following is added to increase the pH of plant-based oil 14: calcium chloride, magnesium sulfide, sodium meta-silicate, sulfated castor oil, ultra-pure H₂O, etc.

In one embodiment, the plant-based oil 14 is olive oil. The olive oil is mixed with an alkaline material at the rate of between one and twenty dl of alkaline material to each gallon of olive oil and optionally stirred while injecting a gas 19 comprising ozone (O₃) until the olive oil, alkaline material, and ozone (O₃) coagulates. In some embodiments, the alkaline material is any one or more materials selected from calcium chloride, magnesium sulfide, sodium meta-silicate, and sulfated castor oil.

In another embodiment, olive oil is mixed with an alkaline material at the rate of between one and twenty dl of an alkaline material to each gallon of olive oil and injecting a gas 19 comprising ozone (O₃) (optionally while being stirred) until the olive oil, alkaline material, and ozone (O₃) coagulates. In this embodiment, the alkaline material is soluble alkali metal silicate with an aqueous medium containing a dissolved substance which is a source of calcium ion and a dissolved substance which is a source of magnesium ion.

In another embodiment, the olive oil is mixed with an alkaline material at the rate of between one and twenty dl of an alkaline material to each gallon of olive oil and injecting a gas 19 comprising ozone (O₃) (optionally while being stirred) until the olive oil, alkaline material, and ozone (O₃) coagulates. In this embodiment, the alkaline material comprises sodium metasilicate with an aqueous medium containing a dissolved substance which is a source of calcium ions and a dissolved substance which is a source of magnesium ions, the aqueous medium containing the dissolved substances in amounts to provide between about 1*10⁴ to 1*10¹ mole per liter each of calcium and magnesium ion, resulting in a molar ratio of calcium ion to magnesium ion of between 2.0:1.0 and 1.0:2.0.

In another embodiment, any plant-based oil 14 or combination of plant-based oils 14 is mixed with an alkaline material at the rate of between one (1) and twenty (20) dl of alkaline material to each gallon of oil and injecting a gas 19 comprising ozone (O₃) (optionally while being stirred) until the plant-based oil(s) 14, alkaline material, and ozone (O₃) coagulates. In some embodiments, the alkaline material is any one or more materials selected from calcium chloride, magnesium sulfide, sodium meta-silicate, and sulfated castor oil.

In another embodiment, a plant-based oil 14 or combination of several plant-based oils 14 is mixed with an alkaline material at the rate of between one (1) and twenty (20) dl of an alkaline material to each gallon of plant-based oil(s) 14 and injecting a gas 19 comprising ozone (O₃) (optionally while being stirred) until the plant-based oil(s) 14, alkaline material, and ozone (O₃) coagulates. In this embodiment, the alkaline material is soluble alkali metal silicate with an aqueous medium containing a dissolved substance which is a source of calcium ion and a dissolved substance which is a source of magnesium ion.

In another embodiment, a plant-based oil 14 or combination of plant-based oils 14 is mixed with an alkaline material at the rate of between one (1) and twenty (20) dl of an alkaline material to each gallon of plant-based oil(s) 14 and injecting a gas 19 comprising ozone (O₃) (optionally while being stirred) until the plant-based oil(s) 14, alkaline material, and ozone (O₃) coagulates. In this embodiment, the alkaline material comprises sodium metasilicate with an aqueous medium containing a dissolved substance which is a source of calcium ions and a dissolved substance which is a source of magnesium ions, the aqueous medium containing the dissolved substances in amounts to provide between about 1*10⁴ to 1*10¹ mole per liter each of calcium and magnesium ion, resulting in a molar ratio of calcium ion to magnesium ion of between 2.0:1.0 and 1.0:2.0.

In another embodiment, a plant-based oil 14 or combination of plant-based oils 14 is mixed with an alkaline material having a pH of approximately 13 at the rate of one teaspoon of the alkaline material to each 32 ounces of plant-based oil(s) 14 and injecting a gas 19 comprising ozone (O₃) (optionally while being stirred) until the plant-based oil(s) 14, the alkaline material, and ozone (O₃) coagulates. In this embodiment, the alkaline material is made by mixing 750 milliliters of ion-depleted H₂O having approximately 17 megohms-cm of resistance (e.g. ultra-pure water having an electrical resistance of 16-26 megohms) with 330 milliliters of calcium chloride, 660 milligrams of magnesium sulfate, 47,500 milligrams of Sodium silicate pentahydrate, and 1000 milligrams sodium benzoate. The mixture is heated to 80 degrees Celsius for ten minutes, then cooled to room temperature (approximately 70 degrees F.) for ten minutes, at which time 100 milliliters of sulfated castor oil is added, then the mixture is heated to 90 degrees Celsius for approximately one hour.

Note that in all embodiments, it is anticipated that the plant-based oil 14 be exposed to the gas 19 with or without stirring, as many gases 19 will coagulate the plant-based oil 14 without stirring after a period of exposure time.

In some embodiments one or more fragrances and/or colorings are added to the plant-based oil(s) 14 and optional alkaline material before or after mixing with the gas 19.

The resulting, coagulated ointment retains the coagulated state with or without refrigeration, though product life is extended when refrigerated.

By ozonating olive oil 14, the ozone is stabilized without adding artificial stabilizers, chemicals, or preservatives. Ozone is bubbled at very high concentrations, under a controlled environment (e.g., for days) until the olive oil 14 coagulates (e.g. solidifies). During this step, a catalytic reaction occurs and potentially three primary, organic peroxides are created through the process. The first peroxide created reacts with the olive oil 14 to produce a second peroxide, and then again to form a long chain: terpene ozonide (C10H18O3). The ozonide level is determined based upon the 3, 6, 9 fatty acid ratio of the olive oil 14. This solid form of olive oil 14 forms a salve-like substance and will keep for many months on the shelf. If kept refrigerated, the ozonated olive oil 14 maintains its full effectiveness much longer.

Equivalent elements can be substituted for the ones set forth above such that they perform in substantially the same manner in substantially the same way for achieving substantially the same result.

It is believed that the system and method as described and many of its attendant advantages will be understood by the foregoing description. It is also believed that it will be apparent that various changes may be made in the form, construction and arrangement of the components thereof without departing from the scope and spirit of the invention or without sacrificing all of its material advantages. The form herein before described being merely exemplary and explanatory embodiment thereof. It is the intention of the following claims to encompass and include such changes.

What is claimed is:

1. A coagulated composition for topical application, the composition comprising:
a plant-based oil mixed with at least 0.01% by weight of an alkaline material; and
a gas, the gas infused into the plant-based oil and the alkaline material.

2. The coagulated composition of claim 1, wherein the alkaline material is one or more materials selected from the group consisting of calcium chloride, magnesium sulfide, sodium meta-silicate, and sulfated castor oil.

3. The coagulated composition of claim 1, wherein the alkaline material has a pH of approximately 13 and comprises a mixture made by mixing 750 milliliters of ion-depleted H₂O having approximately 17 megohms-cm of resistance (e.g. ultra-pure water having an electrical resistance of 16-26 megohms) with 330 milliliters of calcium chloride, 660 milligrams of magnesium sulfate, 47,500 milligrams of Sodium silicate pentahydrate, and 1000 milligrams sodium benzoate which is heated to 80 degrees Celsius for ten minutes, then cooled to room temperature for ten minutes, at which time 100 milliliters of sulfated castor oil is added, then the mixture is heated to 90 degrees Celsius for approximately one hour.

4. The coagulated composition of claim 1, wherein the gas comprises ozone (O₃).

5. The coagulated composition of claim 1, wherein the plant-based oil is one or more oils selected from the group consisting of hemp oil, olive oil, sunflower oil, and jojoba oil.

6. A coagulated composition for topical application, the composition comprising:
a plant-based oil mixed with an alkaline material and exposed to ozone (O₃) until the plant-based oil coagulates.

7. The coagulated composition of claim 6, wherein the alkaline material is one or more materials selected from the group consisting of calcium chloride, magnesium sulfide, sodium meta-silicate, and sulfated castor oil.

8. The coagulated composition of claim 7, wherein the alkaline material is added to the plant-based oil at a rate of from 1 to 20 dl of alkaline material per gallon of plant-based oil.

9. The coagulated composition of claim 6, wherein the alkaline material has a pH of approximately 13 and comprises 750 milliliters of ion-depleted H₂O having approximately 17 megohms-cm of resistance, 330 milliliters of calcium chloride, 660 milligrams of magnesium sulfate, 47,500 milligrams of Sodium silicate pentahydrate, 1000 milligrams sodium, and 100 milliliters of sulfated castor oil.

10. The coagulated composition of claim 9, wherein the alkaline material is added to the plant-based oil at a rate of one teaspoon (4.9289 ml) per 32 ounces (0.946353 liter) of water.

11. The coagulated composition of claim 6, wherein the plant-based oil is one or more oils selected from the group consisting of olive oil, sunflower oil, and jojoba oil.

12. A method of making a coagulated composition for topical application, the method comprising, in sequence:
mixing a mixture of 750 milliliters ultra-pure water having an electrical resistance of 16-26 megohms-cm with 330 milliliters of calcium chloride, 660 milligrams of magnesium sulfate, 47,500 milligrams of Sodium silicate pentahydrate, and 1000 milligrams sodium benzoate;
heating the mixture to 80 degrees Celsius for ten minutes;
cooling the mixture for ten minutes at room temperature (approximately 70 degrees F.);
adding 100 milliliters of sulfated castor oil to the mixture;
heating the mixture to 90 degrees Celsius for approximately one hour;
mixing the mixture with an amount of plant-based oil into a mixing container, the mixture is added to the plant-based oil at a rate of one teaspoon (4.9289 ml) of the mixture per 32 ounces (0.946353) of the plant-based oil;
injecting a gas into the plant-based oil at a location below a surface of the plant-based oil until the plant-based oil coagulates.

13. The method of claim 12, wherein the ultra-pure water is ion-depleted H₂O.

14. The method of claim 13, wherein the ion-depleted H₂O has approximately 17 megohms of resistance per cm.

15. The method of claim 12, wherein the plant-based oil is one or more oils selected from the group consisting of olive oil, sunflower oil, and jojoba oil.

16. The method of claim 12, wherein the gas consists of ozone (O₃).

17. The method of claim 12, wherein the gas comprises ozone (O₃).

18. The method of claim 12, further comprising the step of stirring the plant-based oil during the step of injecting the gas.

19. The method of claim 12, further comprising the step of adding a fragrance to the plant-based oil before the step of injecting the gas is performed.

20. The method of claim 12, further comprising the step of adding a coloring to the plant-based oil before the step of injecting the gas is performed.
